SEQUENCE LISTING (32 sequences)

SEQ ID NO: 1 - protein, 37 aa, Homo sapiens [sequence omitted]

SEQ ID NO: 2 - protein, 37 aa, Artificial Sequence
  Synthetic peptide construct
  MISC_FEATURE: c-term may be amino, alkylamino, dialkylamino, cycloalkylamino, arylamino, aralkylamino, alkyloxy, aryloxy, or aralkyloxy
  MISC_FEATURE (1)..(1): Lys, Ala, Ser, or absent
  MISC_FEATURE (2)..(7): residues 2 and 7 are independently selected amino acid residues having side chains which are chemically bonded to each other to form an intramolecular linkage
  MISC_FEATURE (13)..(13): Ala, Ser, or Thr
  MISC_FEATURE (17)..(17): Val, Leu, or Ile
  MISC_FEATURE (18)..(18): His or Arg
  MISC_FEATURE (19)..(19): Ser or Thr
  MISC_FEATURE (20)..(20): Ser, Thr, Gln, or Asn
  MISC_FEATURE (21)..(21): Asn, Gln, or His
  MISC_FEATURE (23)..(23): Phe, Leu, or Tyr
  MISC_FEATURE (25)..(25): Ala or Pro
  MISC_FEATURE (26)..(26): Ile, Val, Ala, or Leu
  MISC_FEATURE (28)..(28): Ser, Pro, Leu, Ile, or Thr
  MISC_FEATURE (29)..(29): Ser, Pro, or Thr
  MISC_FEATURE (31)..(31): Asn, Asp, or Gln
XXNTATXATQRLXNFLXXXXXNXGXXLXXTXVGSNTY

SEQ ID NO: 3 - protein, 25 aa, Artificial Sequence
  Synthetic peptide construct
  MISC_FEATURE: N-term may be hydrogen or an acetyl group
  MISC_FEATURE (1)..(1): Ala or absent
  MISC_FEATURE (4)..(4): Arg, Gln, Lys, Asn, or Leu
  MISC_FEATURE (8)..(8): Gln, Glu, Asn, Asp, or Phe
  MISC_FEATURE (22)..(22): Ala or Ser
XTQXLANXLVRLQTYPRTNVGXNTY

SEQ ID NO: 4 - protein, 36 aa, Artificial Sequence
  Synthetic peptide construct
CNTATCATQRLANFLVHSSNNFGAILSSTNVGSNTY

SEQ ID NO: 5 - protein, 37 aa, Artificial Sequence
  Synthetic peptide construct
KCNTATCATQRLANFLVHSSNNFGAILPSTNVGSNTY

SEQ ID NO: 6 - protein, 37 aa, Artificial Sequence
  Synthetic peptide construct
KCNTATCATQRLANFLVHSSNNFGPILPPTNVGSNTY

SEQ ID NO: 7 - protein, 37 aa, Artificial Sequence
  Synthetic peptide construct
KCNTATCATQRLANFLVRSSNNFGPILPSTNVGSNTY

SEQ ID NO: 8 - protein, 36 aa, Artificial Sequence
  Synthetic peptide construct
CNTATCATQRLANFLVHRSNNFGPILPSTNVGSNTY

SEQ ID NO: 9 - protein, 37 aa, Artificial Sequence
  Synthetic peptide construct
KCNTATCATQRLANFLVHSSNNFGPVLPPTNVGSNTY

SEQ ID NO: 10 - protein, 37 aa, Artificial Sequence
  Synthetic peptide construct
KCNTATCATQRLANFLVRSSNNFGPILPPTNVGSNTY

SEQ ID NO: 11 - protein, 36 aa, Artificial Sequence
  Synthetic peptide construct
CNTATCATQRLANFLVRSSNNFGPILPPSNVGSNTY

SEQ ID NO: 12 - protein, 36 aa, Artificial Sequence
  Synthetic peptide construct
CNTATCATQRLANFLVHSSNNFGPILPPSNVGSNTY

SEQ ID NO: 13 - protein, 37 aa, Artificial Sequence
  Synthetic peptide construct
KCNTATCATQRLANFLVHSSNNLGPVLPPTNVGSNTY

SEQ ID NO: 14 - protein, 37 aa, Artificial Sequence
  Synthetic peptide construct
KCNTATCATQRLANFLVHSSNNLGPVLPSTNVGSNTY

SEQ ID NO: 15 - protein, 36 aa, Artificial Sequence
  Synthetic peptide construct
CNTATCATQRLANFLVHSSNNLGPVLPSTNVGSNTY

SEQ ID NO: 16 - protein, 37 aa, Artificial Sequence
  Synthetic peptide construct
KCNTATCATQRLANFLVRSSNNLGPVLPSTNVGSNTY

SEQ ID NO: 17 - protein, 37 aa, Artificial Sequence
  Synthetic peptide construct
KCNTATCATQRLANFLVRSSNNLGPILPPTNVGSNTY

SEQ ID NO: 18 - protein, 37 aa, Artificial Sequence
  Synthetic peptide construct
KCNTATCATQRLANFLVRSSNNLGPILPSTNVGSNTY

SEQ ID NO: 19 - protein, 37 aa, Artificial Sequence
  Synthetic peptide construct
KCNTATCATQRLANFLIHSSNNLGPILPPTNVGSNTY

SEQ ID NO: 20 - protein, 37 aa, Artificial Sequence
  Synthetic peptide construct
KCNTATCATQRLANFLIHSSNNFGPILPPTNVGSNTY

SEQ ID NO: 21 - protein, 36 aa, Artificial Sequence
  Synthetic peptide construct
CNTATCATQRLANFLIHSSNNLGPILPPTNVGSNTY

SEQ ID NO: 22 - protein, 37 aa, Artificial Sequence
  Synthetic peptide construct
KCNTATCATQRLANFLIRSSNNLGAILSSTNVGSNTY

SEQ ID NO: 23 - protein, 37 aa, Artificial Sequence
  Synthetic peptide construct
KCNTATCATQRLANFLIRSSNNLGAVLSPTNVGSNTY

SEQ ID NO: 24 - protein, 37 aa, Artificial Sequence
  Synthetic peptide construct
KCNTATCATQRLANFLIRSSNNLGPVLPPTNVGSNTY

SEQ ID NO: 25 - protein, 37 aa, Artificial Sequence
  Synthetic peptide construct
KCNTATCATQRLTNFLVHSSHNLGAALLPTDVGSNTY

SEQ ID NO: 26 - protein, 37 aa, Artificial Sequence
  Synthetic peptide construct
KCNTATCATQRLTNFLVHSSHNLGAALSPTDVGSNTY

SEQ ID NO: 27 - protein, 36 aa, Artificial Sequence
  Synthetic peptide construct
CNTATCATQRLTNFLVHSSHNLGAALPSTDVGSNTY

SEQ ID NO: 28 - protein, 37 aa, Artificial Sequence
  Synthetic peptide construct
KCNTATCATQRLTNFLVRSSHNLGAALSPTDVGSNTY

SEQ ID NO: 29 - protein, 37 aa, Artificial Sequence
  Synthetic peptide construct
KCNTATCATQRLTNFLVRSSHNLGAILPPTDVGSNTY

SEQ ID NO: 30 - protein, 37 aa, Artificial Sequence
  Synthetic peptide construct
KCNTATCATQRLTNFLVRSSHNLGPALPPTDVGSNTY

SEQ ID NO: 31 - protein, 25 aa, Artificial Sequence
  Synthetic peptide construct
VLGKLSQELHKLQTYPRTNTGSGTP

SEQ ID NO: 32 - protein, 25 aa, Artificial Sequence
  Synthetic peptide construct
  MOD_RES (1)..(1): ACETYLATION
VLGKLSQELHKLQTYPRTNTGSNTY

(19) United States
(12) Patent Application Publication
(10) Pub. No.: US 2007/0275877 A1
(43) Pub. Date: Nov. 29, 2007

(54) Methods for Treating or Ameliorating Ghrelin-Associated Diseases and Disorders

(75) Inventors: Alain Baron, San Diego, CA (US); Andrew A. Young, San Diego, CA (US); Bronislava Gedulin, San Diego, CA (US)

Correspondence Address: Intellectual Property Department;Amylin Pharmaceuticals, Inc., 9360 Towne Centre Drive, San Diego, CA, 92121, US

(73) Assignee: AMYLIN PHARMACEUTICALS, INC., SAN DIEGO, CA (US)

(21) Appl. No.: 10/569,771

(22) PCT Filed: Aug. 30, 2004
(86) PCT No.: PCT/US04/28283

§ 371 Date: Feb. 24, 2006

Related U.S. Application Data
(60) Provisional application No. 60/498,898, filed on Aug. 29, 2003.
(60) Provisional application No. 60/554,528, filed on Mar. 18, 2004.

(51) Int. Cl. A61K 38/17; A61K 31/00; A61K 38/28

(52) U.S. Cl. 514/3; 514/1; 514/12

(57) ABSTRACT

Methods for modulating the effective levels of ghrelin are disclosed. These methods include the use of amylin, amylin agonists and amylin antagonists to regulate the effective levels of ghrelin. Methods for the prevention, treatment, or amelioration of ghrelin-associated diseases or disorders utilizing the methods for modulating ghrelin are also disclosed.

### RELATED APPLICATIONS

This application claims the benefit of priority to U.S. Provisional Patent Application Ser. Nos. 60/498,898 and 60/554,528, filed Aug. 29, 2003 and Mar. 18, 2004, respectively, and incorporates in their entirety the contents thereof.

### FIELD OF THE INVENTION

The present invention relates to the medical field and in particular to the field of ghrelin-associated disease and disorders.

### BACKGROUND OF THE INVENTION

Ghrelin is a 28 amino acid peptide hormone, discovered in 1999, and found to be an endogenous ligand for growth hormone secretagogue receptor (GHS-R) and stimulates growth hormone (GH) release from the pituitary cells (Kojima et al. 1999 Nature 402(6762):656-60). It has been reported that injecting people with ghrelin led to a significant, prolonged increase in circulating growth hormone concentrations (Arvat, et al. 2000 J. Endocrinol Invest 23(8):493-5).

The understanding of the hormone ghrelin has evolved from an endogenous growth hormone secretagogue to a regulator of energy balance, a pleiotropic hormone with multiple sources, numerous target tissues and most likely several physiological functions. Horvath et al. 2003 Curr Pharm Des. 9(17):1383-95. Ghrelin has also been linked to diabetes and cardiovascular disease.

One function of ghrelin under intense investigation is its role in energy utilization. Ghrelin has been reported to have an orexigenic effect, a weight regulatory effect, as well as an effect on adiposity.

Studies suggest that ghrelin is an appetite stimulant, i.e., ghrelin increases food intake. Ghrelin's orexigenic effect is supported by studies of peripheral administration of ghrelin in humans (Wren et al. 2001 J Clin Endocrinol Metab 86(12):5992) and central administration in animals (Wren et al. 2001 Diabetes 50(11):2540-7). One proposed mechanism of ghrelin's action is that ghrelin increases the hypothalamic neuropeptide Y and Agouti-related protein mRNA levels. Neuropeptide Y and Agouti-related protein are orexigenic neuropeptides, that can cause increased food intake and increased body weight (Kamegai et al. 2001 Diabetes 50(11):2438-43; Shintani et al. 2001 Diabetes 50:227-232). Consistent with these findings is the observation that antagonists of ghrelin appear to reduce food intake and body weight gain in mice (Asakawa et al. 2003 Gut 52(7):947-52).

Ghrelin has been reported to reduce fat utilization in adipose tissue in rodents (Tschop et al. 2000 Nature 407:908-13) as well as be involved in rat adipogenesis (Choi et al. 2003 Endocrinology 144(3):754-9).

Ghrelin is thought to be involved in weight regulation although its role is not fully understood. In general, plasma ghrelin concentrations appear to vary reciprocally with nutritional state, i.e., high when nutrient availability is low and low when nutrient availability is high (Shiiya et al. 2002 J Clin Endocrinol Metab 87(1):240-4). For example, ghrelin concentrations have been reported to increase during fasting (Asakawa et al. 2001 Gastroenterology 120(2):337-45) and chronic food restriction (Gualillo et al. 2002 Obes Res 10(7):682-7; Ravussin et al. 2001 J Clin Endocrinol Metab 86(9):4547-51). Ghrelin concentrations have been reported to decrease with hyperglycemia or a glucose challenge (Shiiya et al. 2002 J Clin Endocrinol Metab 87(1):240-4; Cappiello et al. 2002 Eur J Endocrinol 147( 2):189-94; Nakagawa et al. 2002 Clin Sci (Lond) 103(3):325-8; McCowen et al. 2002 J Endocrinol 175(2):R7-R11) and with feeding (Tolle et al. 2002 Endocrinology 143(4):1353-61). Thus, ghrelin is thought to be a hunger signal, prompting the subject to eat when nutrient availability is low. Ghrelin concentrations have been reported to increase upon dieting, suggesting that the increased ghrelin concentration is signaling the body to increase food intake to maintain body weight.

Ghrelin concentrations are reported to be lower in people who are obese than in people of normal weight (Rosicka et al. 2003 Physiol Res 52(1):61-6); however, it has also been reported that obese people do not show a postprandial decrease in ghrelin levels as seen in normal weight people (English et al. 2002 J Clin Endocrinol Metab 87(6):2984). People with anorexia nervosa are reported to have higher than normal levels of ghrelin (Tanaka et al. 2003 Psychoneuroendocrinology 28(7):829-35); although, it has also been reported that their levels of ghrelin do decrease with weight gain (Otto et al. 2001 Eur J Endocrinol 145(5):669-73).

Studies on the relationship between ghrelin and insulin have not been consistent. For example, while some studies have shown that ghrelin stimulates insulin secretion in humans and rats (Lee et al. 2002 Endocrinology 143(1): 185-90; Date et al. 2002 Diabetes 51(1): 124-9) as well as normal and diabetic rats (Adeghate et al. 2002 J Neuroendocronol 14(7):555-60), other studies report that ghrelin reduces insulin secretion in humans and mouse (Broglio et al. 2001 J Clin Endocrinol Metab 86(10):5083-6; Egido et al. 2002 Eur J Endocriol 146(2):214-4; Reimer et al. 2003 Endocrinology 144(3):916-21. Moreover, while some report insulin decreases circulating levels of ghrelin (Flanagan et al. 2003 Am J Physiol Endocrinol Metab 284(2):E313-E316; Saad et al. 2002 J Clin Endocrinol Metab 87(8):3997-4000), others report that insulin does not regulate ghrelin levels (Schaller et al. 2003 Diabetes 52(1):16-20).

Ghrelin has been reported to induce vasodilation, improve left ventricular dysfunction and attenuate cardiac cachexia in rats with chronic heart failure (CHF) (Nagaya, et al. 2001 Circulation 104:1430) as well as having a beneficial hemodynamic effect in human patients with CHF (Nagaya et al. 2001 J Clin Endocrin & Metab 86(12):5854-59).

To date, most of the research has centered around using ghrelin for treating diseases or disorders, finding agonists of ghrelin to increase the level of ghrelin activity or antagonists of ghrelin to oppose the actions of ghrelin, such as described in WO 01/92292, WO 01/87335 and U.S. Patent Application No. US2002/0187938. A similar approach is taught by U.S. Patent Application No. 2001/0020012, where ligands for the receptor GHS-R 1A are used to regulate food intake.

What is described herein are novel methods for regulating effective ghrelin levels in a subject as well as methods for treating, preventing, or ameliorating ghrelin-associated diseases and disorders.

### SUMMARY OF THE INVENTION

In one general aspect, methods of the invention include inhibiting ghrelin secretion in an individual comprising administering to said individual an exogenous amylin, amylin analog, or amylin agonist, or comprising increasing endogenous levels of amylin. In another general aspect, methods of the invention include reducing endogenous levels of ghrelin in an individual comprising administering to said individual an exogenous amylin, amylin analog, or amylin agonist, or comprising increasing endogenous levels of amylin. In certain embodiments, the endogenous levels of amylin can be increased with the administration of an insulinotropic agent. Exemplary insulinotropic agents include, but are not limited to, a GLP-1, an exendin, or an analog, derivative, or agonist of a GLP-1 or exendin, or a sulfonylurea. In still another general aspect, compounds that interfere with or enhance the ability of amylin to affect or bind to receptors in the area postrema (AP) can be used regulate ghrelin levels.

In certain embodiments, methods of the invention include treating, preventing or ameliorating ghrelin-associated diseases or disorders that can be benefited by a reduction in ghrelin levels by the above described methods. Examples of such ghrelin-associated diseases or disorders include, but are not limited to, Prader-Willi syndrome or diabetes mellitus and its complications. Also contemplated in the invention are methods to treat reduce, or prevent from worsening conditions caused or enhanced by ghrelin, such as obesity, hyperphagia, hyperlipidemia, or other disorders associated with hypernutrition, as well as other conditions known in the art.

In other embodiments, methods of the invention can be used to treat, prevent, or ameliorate a ghrelin-associated disease or disorder that is related to increased growth hormone levels, such as acromegaly or diabetes mellitus, among others.

In still other embodiments, use of the methods of the invention may further include insulin or glucose (or a glucose source) to assist in the inhibition of ghrelin secretion.

In yet another general aspect, methods of the invention include increasing endogenous levels of ghrelin in an individual comprising administering to said individual an amylin antagonist or a compound that decreases the effective levels of amylin, such as antibodies. In still another general aspect, methods of the invention include increasing ghrelin secretion in an individual comprising administering to said individual an amylin antagonist.

In certain embodiments, methods of the invention can be used to treat, prevent or ameliorate ghrelin-associated diseases or disorders that can be benefited by an increase in ghrelin levels. Examples of such diseases and disorders include, but are not limited to, anorexia nervosa, bulimia, cachexia, including cachexia of cancer, AIDS, and wasting, including wasting in the elderly. Methods of the invention also include stimulating ghrelin to increase food intake or increase release of growth hormones. Thus, methods of the invention can be used to treat conditions characterized by decreased growth hormone levels such as those described above as well as children of short stature, muscle wasting and aging.

Therefore, methods of the invention include modulating, or otherwise affecting, ghrelin levels in an individual. The invention further contemplates uses of the compounds described herein in the manufacture of a medicament for modulating ghrelin levels. The medicament may be used in the treatment of any ghrelin-associated diseases or disorders.

The details of one or more embodiments of the invention are set forth in the accompanying drawings and the description below. Other features, objects, and advantages of the invention will be apparent from the description and drawings, and from the claims. All references cited herein are incorporated by reference.

### BRIEF DESCRIPTION OF THE DRAWINGS

FIG. 1 depicts a graph of the effect of exogenously administered rat amylin on endogenous ghrelin levels in an animal model. 125 μg pentagastrin (PG) was administered at t=0 and 10 μg amylin was administered at t=20 (dotted lines).

FIGS. 2A and 2B depict graphs of the effect of rat amylin on ghrelin levels in an animal model in the presence and absence of pentagastrin.

FIGS. 3A and 3B depict graphs of dose-response effects of rat amylin on ghrelin levels in an animal model.

FIGS. 4A and 4B depict graphs of the effects of an amylin receptor antagonist on ghrelin levels in an animal model.

FIG. 5 depicts a graph of the effect of the area postrema on amylin's effect on ghrelin secretion.

FIG. 6 depicts a graph of the effect of an amylin analog on ghrelin levels in humans.

### DESCRIPTION OF THE INVENTION

It has now been discovered that amylin, amylin agonist analogs and derivatives, and amylin agonists (including calcitonins, calcitonin gene-related peptides, and analogs thereof) can decrease ghrelin levels. It has further been found that an amylin antagonist can increase ghrelin levels. It appears that modulation of the effective levels of amylin, with amylin, amylin agonists, amylin antagonists, or other compounds that decrease the effective level of amylin such as antibodies, may inhibit, or stimulate in the case of antagonists and antibodies, ghrelin secretion. The present invention is directed to modulating endogenous levels of ghrelin by either increasing the effective level of amylin or amylin agonists in the body, by direct or indirect means, or by decreasing the effective level of amylin using amylin antagonists or inhibiting amylin production. The phrase “effective level,” refers to the level of the desired activity of the molecules and not necessarily limited to the number of molecules. For example, the effective level of amylin may be decreased to stimulate ghrelin secretion by using amylin antagonists, without a necessary concomitant decrease in the amount of free amylin present in a subject.

An example of a direct means for increasing the effective level of an amylin, an amylin agonist, or for decreasing the effective level of amylin by an amylin antagonist is by administration of the amylin, amylin agonist, or amylin antagonist as a peptide, a prodrug, or as pharmaceutical salts thereof, to the body. The term “prodrug” refers to a compound that is a drug precursor which, following administration, releases the drug in vivo via some chemical or physiological process, for example, proteolytic cleavage, or upon reaching an environment of a certain pH. An example of an indirect means for increasing the effective level of amylin is to induce amylin production by gene therapy, e.g., the introduction to a body of amylin producing cells, or stimulating beta cells to produce more amylin, e.g., by administering an agent such as a GLP-1, an exendin such as exendin-4 (exenatide), or a sulfonylurea. Also contemplated as beta-cell stimulating agents are analogs, derivatives, and agonists of GLP-1 and exendin. Examples of such agents can be found in U.S. Pat. No. 5,512,549; U.S. Pat. No. 5,574,008; U.S. Pat. No. 5,545,618; U.S. Pat. No. 6,528,486; U.S. Pat. No. 5,614,492; WO 9746584; U.S. Pat. No. 5,424,286; U.S. Pat. No. 6,593,295; International Application No. PCT/US98/16387, filed Aug. 6, 1998, claiming the benefit of U.S. Provisional Patent Application Ser. No. 60/055,404, entitled, filed Aug. 8, 1997; International Application No. PCT/US98/24210, filed Nov. 13, 1998, claiming priority on U.S. Provisional Patent Application Ser. No. 60/065,442, filed Nov. 14, 1997; and International Application No. PCT/US98/24273, filed Nov. 13, 1998, claiming priority on U.S. Provisional Patent Application Ser. No. 60/066,029, filed Nov. 14, 1997; U.S. Provisional Application Ser. No. 60/132,018, filed Apr. 30, 1999; U.S. Patent Application No. 20030087821; U.S. Patent Application No. 20030087820; and U.S. Patent Application Ser. No. 20010047084; all of which are incorporated herein by reference. An example of an indirect means for decreasing the effective level of amylin is inhibiting amylin production by antisense technology or RNAi technology.

Moreover, amylin acts at the area postrema, a circumventricular organ that is densely populated with amylin receptors. Without wishing to be bound by theory, it is believed that the amylin regulates ghrelin secretion via the area postrema.

Methods of the invention can be used on any individual in need of such methods. These individuals may be any mammal including, but not limited to, humans, dogs, horses, cows, pigs, and other commercially valuable or companion animals.

### EXAMPLES

The following methods apply to Examples 1-4 as indicated. Male Harlan Sprague Dawley® (HSD) rats were housed at 22.8±0.8° C. in a 12:12 hour light:dark cycle. All experiments were performed in the light cycle. Animals were fasted for approximately 20 hours before experimentation (examples 1, 2 and 3) or fed ad libidum (Example 4). All animals were given free access to water until the start of the experiment. The animals' tails were anesthetized with 20% benzocaine (Hurricaine, Beutlich Pharmaceutical, Waukegan, Ill.), and blood samples were collected from the tail vein. Total and active ghrelin concentrations were measured using Linco RIA kits GHRA-89HK and GHRA-88HK, respectively.

### Example 1

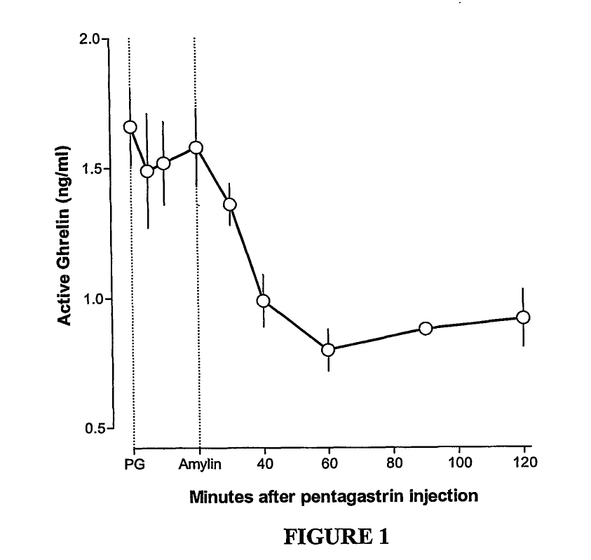

HSD rats were subjected to periodic blood sampling from the topically anesthetized tail and ghrelin levels were assayed. At t=0, rats (n=6) were injected s.c. with 125 μg/kg pentagastrin (Sigma) to stimulate gastric acid secretion (PG=0 min in FIG. 1), and 20 min later were injected subcutaneously (s.c.) with 10 μg rat amylin. The blood samples were analyzed for total and active (acylated) ghrelin (Linco). As shown in FIG. 1, amylin reduced active ghrelin by 50% within 1 hour.

### Example 2

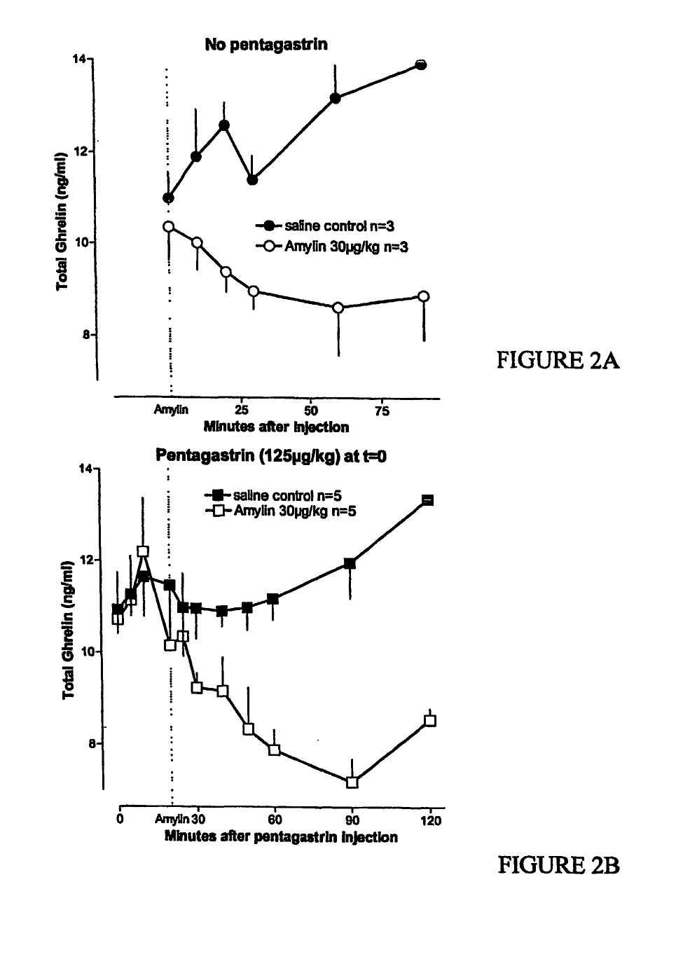

These experiments were conducted to examine whether exogenous amylin inhibits ghrelin secretion independent of pentagastrin stimulation. Fasted rats were given either a subcutaneous injection of saline or of 30 μg/kg rat amylin (FIG. 2A) or a subcutaneous injection of either saline or 125 μg/kg pentagastrin (Sigma, Lot#050K1525) (FIG. 2B) at time=0 min. In the experiments shown in FIG. 2B, rat amylin (AC0128, lot #AR2081-42A, Amylin Pharmaceuticals), at a dose of 30 μg/kg in 100 μl of saline, or saline vehicle alone (n=5,5 respectively), was given by subcutaneous injection at time 20 min. Blood plasma samples were collected at least at times 0, 10, 20, 30, 60, and 90 min. Both FIGS. 2A and 2B show a reduction in total plasma ghrelin with the administration of amylin compared to the saline control, and FIG. 2B confirms that plasma ghrelin was reduced compared to the control in the presence of amylin alone, i.e., without pentagastrin. Pentagastrin appears to enhance the ghrelin lowering effect of amylin.

### Example 3

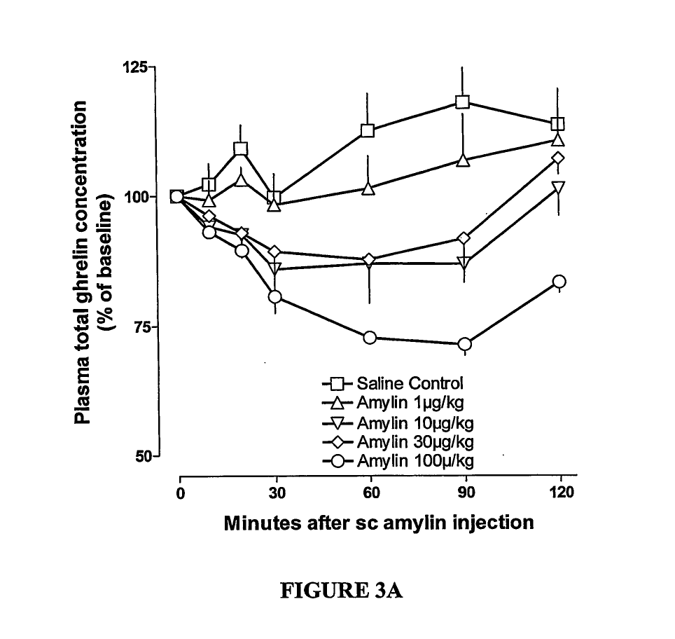

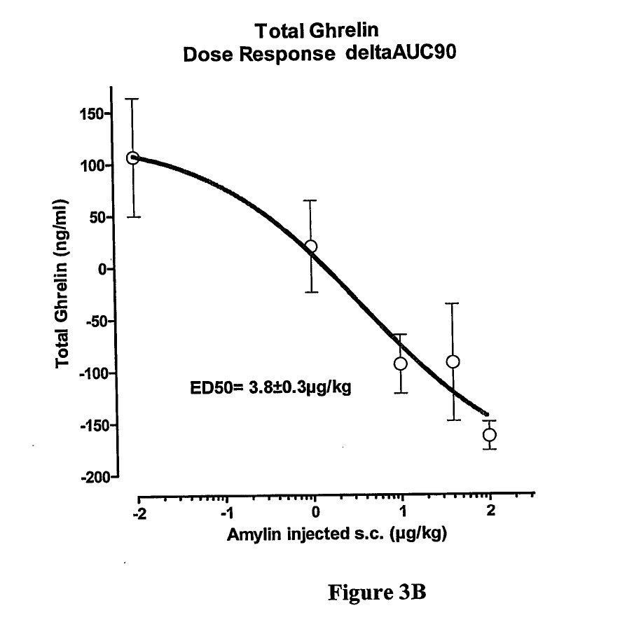

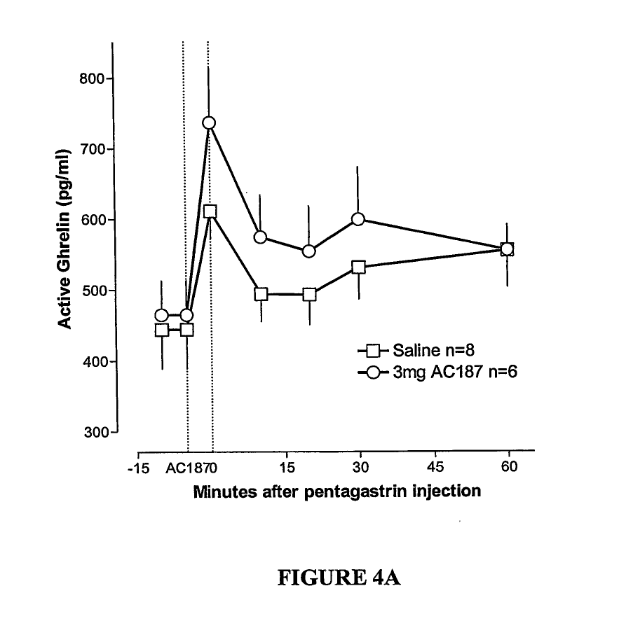

These experiments were conducted to identify dose-response effects of amylin on ghrelin levels in fasted rats. Rat amylin (lot #AR2081-42A, Amylin Pharmaceuticals) in 100 μl saline was given by subcutaneous injections at doses: 0, 1, 10, 30 and 100 μg/kg (n=5, 4, 5, 5, 4 respectively) at time=0 min. Blood plasma samples were collected at time=0, 10, 20, 30, 60, 90 and 120 min after amylin injection. As shown in FIG. 3A, amylin caused a dose-dependent reduction in total plasma ghrelin concentrations, with concentration nadirs occurring 60-90 min after amylin injection. Pairwise statistical analyses were performed using Student's t-test (Instat v2.0, GraphPad Software Inc, San Diego). Results are reported as mean±standard error of the mean, and P<0.05 is used as the level of significance. Reduction of ghrelin was statistically significant with amylin doses of 10 μg/kg and above, and a trend to inhibit ghrelin was apparent with an amylin dose of 1 μg/kg, a potency consistent with this being a physiologic effect of endogenous amylin. Dose-response analysis of amylin inhibition of ghrelin secretion, see FIG. 3B, indicates that this effect may prevail at physiologic amylin concentrations.

The results of these experiments indicate that amylin may be a physiologic inhibitor of ghrelin secretion. Elevated ghrelin and growth hormone (GH) secretion during β-cell deficiency may be at least partly attributable to a lack of amylinergic suppression.

### Example 4

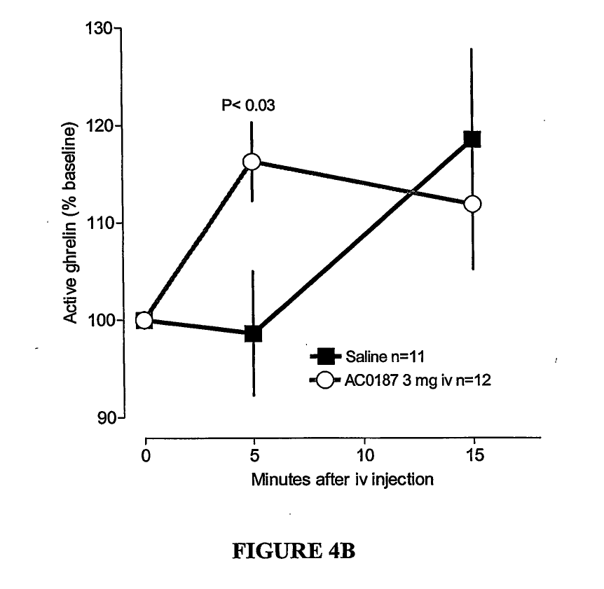

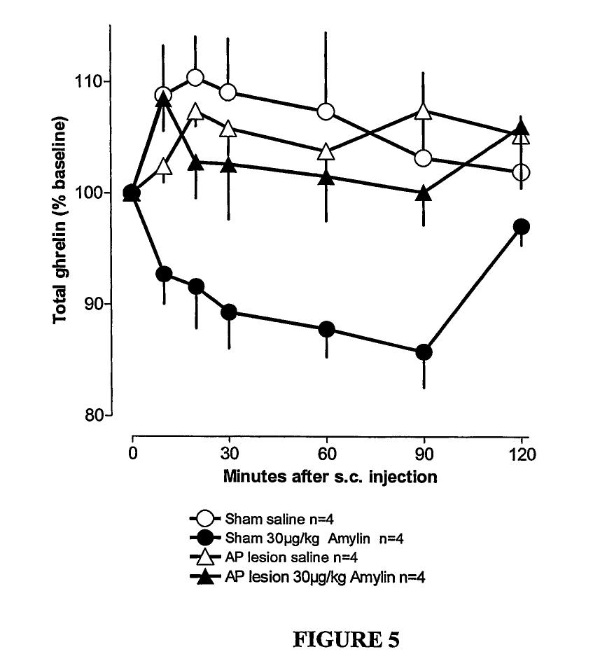

These experiments were conducted to determine whether the amylin receptor antagonist AC187 blocked an effect of endogenous amylin on plasma ghrelin levels in non-fasted rats. Baseline blood samples for ghrelin to be assayed were taken at t=−10 min. AC187 (lot #AR741-72, Amylin Pharmaceuticals), at a dose of 3 mg/rat in 300 μl of saline, or saline vehicle alone, was given by subcutaneous injection (n=6, 8 respectively) at t=−5 min. Animals additionally received a subcutaneous injection of pentagastrin (Sigma, Lot#050K1525) in the amount of 125 μg/kg body mass at time=0 min. Plasma blood samples were collected at time −10, 0, 10, 20, 30, and 60 min. As shown in FIG. 4A, active ghrelin concentrations were higher following administration of AC187 compared to saline injected controls. This experiment indicates that reducing the effective levels of amylin in vivo leads to a decrease in the active levels of plasma ghrelin in vivo.

In a second, related experiment, baseline blood samples for ghrelin to be assayed were taken at t=0 minutes. AC187 (lot#AR2237-18B, Amylin Pharmaceuticals, Inc.), at a dose of 3 mg/rat in 300 μl of saline or saline vehicle alone, was given by intravenous injection in the tail vein at t=0. Plasma samples were collected at time 0, 5, and 15 minutes. Five minutes after administration of AC187 in fed animals, active ghrelin concentrations increased by 16.3±4.0%, as compared to a 1.4±6.4% decrease in saline controls (P<0.03), see FIG. 4B.

### Example 5

In this experiment, fasted, Sprague-Dawley® rats with localized lesions of the area postrema (AP) made by vacuum aspiration 3 weeks before the experiments and sham operated rats (sham) were injected s.c. with amylin (30 μg/kg) or saline (n=4, 4 respectively). At t=0, 10, 20, 30, 60, 90 and 120 minutes after the s.c. injection, total ghrelin concentrations were measured and reports as % of the baseline.

Twenty minutes after a 30 μg/kg subcutaneous amylin injection in Sham rats, active ghrelin concentration decreased by 31.3±3.3%, while active ghrelin concentrations in saline controls increased by 23.5±22.1% (P<0.05). In AP-lesioned rats, there was no significant difference in ghrelin concentration in those receiving amylin versus those receiving saline (increase of 13.7±17.6% vs. +49.6±21.0%; P=0.24). The effect of the AP lesion on amylin inhibition of ghrelin secretion indicates that amylin may act on ghrelin secretion, at least, via the AP.

### Example 6

Ghrelin levels were measured in a single-center, randomized, double-blind, placebo-controlled, two-period, crossover study was designed to examine the acute effects of pramlintide, an amylin analog, in humans. The study consisted of two treatment periods (Period 1/Visit 2 and Period 2/Visit 3) with at least 72 h between each treatment period. After successful completion of all Screening procedures (including informed consent), subjects (n=15) were randomly assigned to one of two treatment sequences (pramlintide: placebo or placebo: pramlintide). Subjects were instructed to fast from 2200 h on the evening before both treatment periods until the start of the test (the next morning).

On the mornings of Period 1 and Period 2, an intravenous cannula was placed in an antecubital vein for blood sampling (t=−1 h). Vital signs and weight were measured at this time. During each treatment period, subjects received study medication (pramlintide or placebo) immediately followed by a standardized, liquid preload meal, which was to be consumed within 3 min. Subjects received a single SC dose of pramlintide 30 μg or placebo. One hour later (t=1 h), subjects consumed an ad libitum buffet meal, which was offered for 45 min. The buffet meal included a selection of carbohydrate-rich foods in quantities in excess of what subjects were expected to eat. Venous blood samples were collected over a 5.5-h period (t=−0.5 h to 5 h) for the measurement of postprandial metabolic and hormonal responses. Procedures for Period 2 were exactly as those described for Period 1, only the alternate treatment was given.

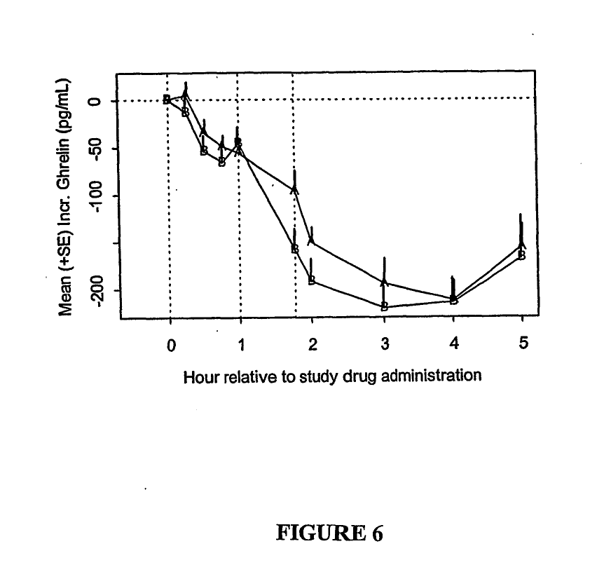

FIG. 6 shows “A” points that are the results from placebo administration and “B” points that are the results from pramlintide administration. FIG. 6 shows that a single administration of pramlintide further reduced the ghrelin levels in subjects following an ad libitum buffet meal compared to those subjects given a placebo. This study also showed pramlintide reduced total caloric intake of the buffet meal, with proportionate reductions in fat, carbohydrate, and protein intake, and reduced duration of the meal time as compared to placebo.

Ghrelin-Associated Diseases and Disorders

The phrase “ghrelin-associated diseases and disorders” refers to any condition that can be treated, prevented or ameliorated through the modulation of ghrelin activity. These include conditions that are enhanced, exacerbated or stimulated by ghrelin, for example, growth hormone release or drive to eat.

While much research has been conducted around ghrelin and its action, because it was only discovered in 1999, much more information about ghrelin and diseases and disorders associated with ghrelin is expected to be discovered as time progresses. To the extent that those new diseases and disorders can be treated, prevented or ameliorated with the regulation of ghrelin activity, the methods of the present invention can be utilized with respect to those conditions.

It is currently believed that the physiological actions of ghrelin include stimulation of growth hormone release, as well as stimulation of hormone secretion from lactotrophs and corticotrophs, orexigenic and cardiovascular actions, anti-proliferative effects on thyroid and breast tumors and regulation of gastric motility and acid secretion through vagal mediation (Ukkola, O et al., 2002, Ann. Med. 34:102-108). By considering the actions of ghrelin, one of ordinary skill in the art will understand the types of diseases or disorders that may be treated, prevented or ameliorated with methods of the present invention.

By way of example only, the types of conditions that can be benefited by a decrease in effective levels of ghrelin include those associated with elevated ghrelin concentrations, such as with type 1 diabetes mellitus, late-stage type 2 diabetes or Prader-Willi syndrome, facilitating decreased food intake, facilitating weight loss, facilitating weight maintenance, and treating obesity. Such benefits may also prevail in patients who do not necessarily exhibit elevations of ghrelin, such as those with obesity or Syndrome X.

Further examples include preventing or ameliorating features and complications of diabetes such as retinopathy, insulin resistance, and dawn phenomenon (a condition characterized by a significant rise in their early morning blood glucose values), and those associated with excessive growth hormone secretion. Methods of the invention can be used to treat other conditions resulting from high levels of growth hormones, such as acromegaly, and for treating cardiovascular disorders. These and other conditions are described in WO 03/051389, WO 01/92292, U.S. Patent Application No. 2002/0187938, U.S. Patent Application No. 2002/0020012, WO 01/87335, which are incorporated herein by reference.

Further, by way of example only, the types of conditions that can be benefited by an increase in effective levels of ghrelin include treating a growth hormone deficient state or any other condition where an anabolic effect is desired, for example, body building. Benefits provided by the methods of the invention include increasing muscle mass, increasing bone density, treating sexual dysfunction, increasing food intake, facilitating weight gain, facilitating weight maintenance, and facilitating recovery of physical function (e.g., after surgery). Some of these conditions are ones that healthy individuals may desire and obtain by using the methods of the invention, such as increased body mass and increased bone density. Other examples of diseases or disorders to be treated or ameliorated include anorexia, bulimia, cachexia, including cachexias of cancer, AIDS, and wasting (e.g., wasting in the elderly). These and other conditions are described in U.S. Pat. No. 6,548,501, U.S. Patent Application No. 2001/0020012, WO 01/92292, and EP 1 166 778, which are incorporated herein by reference.

An exemplary use of the present invention is in the treatment of Prader-Willi syndrome. People who suffer from Prader-Willi suffer from slowed development, severe obesity and an insatiable appetite. Their hunger is so strong that it often requires custodial enforcement of food availability to avert early death as a result of hyperphagia. Ghrelin concentrations in these people are higher than normal by ˜3 to 4-fold. The methods of the invention can be used to help patients with Prader-Willi syndrome reduce their ghrelin levels to more normal levels, curb their appetite, and/or ameliorate other manifestations of this disorder.

Amylin, Amylin Agonists, Amylin Antagonists

Amylin is a 37 amino acid peptide hormone that is co-secreted with insulin from pancreatic beta-cells in response to nutrient stimuli. Human amylin has the following amino acid sequence:

Lys-Cys-Asn-Thr-Ala-Thr-Cys-Ala-Thr-Gln Arg-Leu-Ala-Asn-Phe-Leu-Val-His-Ser-Ser-Asn-Asn-Phe-Gly-Ala-Ile-Leu-Ser-Ser-Thr-Asn-Val-Gly-Ser-Asn-Thr-Tyr (SEQ ID NO:1), although the use of amylins from any species is contemplated.

It has surprisingly been found that modulation of effective amylin levels in vivo such as through the use of amylin, amylin agonists, and amylin antagonists, can modulate the effective levels of ghrelin in vivo.

Amylin agonists contemplated in the use of the invention include those described in U.S. Pat. Nos. 5,686,411, 6,114,304, and 6,410,511, which are herein incorporated by reference in their entirety. Such compounds include those having the formula ¹A₁-X-Asn-Thr-⁵Ala-Thr-Y-Ala-Thr-¹⁰Gln-Arg-Leu-B₁-Asn-¹⁵Phe-Leu-C₁-D₁-E₁-²⁰F₁-G₁-Asn-H₁-Gly-²⁵I₁-J₁-Leu-K₁-L₁-³⁰Thr-M₁-Val-Gly-Ser-³⁵Asn-Thr-Tyr-Z (SEQ ID NO:2)
wherein A₁ is Lys, Ala, Ser or hydrogen;
B₁ is Ala, Ser or Thr;
C₁ is Val, Leu or Ile;
D₁ is His or Arg;
E₁ is Ser or Thr;
F₁ is Ser, Thr, Gln or Asn;
G₁ is Asn, Gln or His;
H₁ is Phe, Leu or Tyr;
I₁ is Ala or Pro;
J₁ is Ile, Val, Ala or Leu;
K₁ is Ser, Pro, Leu, Ile or Thr;
L₁ is Ser, Pro or Thr;
M₁ is Asn, Asp, or Gln;

X and Y are independently selected amino acid residues having side chains which are chemically bonded to each other to form an intramolecular linkage; and

Z is amino, alkylamino, dialkylamino, cycloalkylamino, arylamino, aralkylamino, alkyloxy, aryloxy or aralkyloxy.

Suitable side chains for X and Y include groups derived from alkyl sulfhydryls which may form disulfide bonds; alkyl acids and alkyl amines which may form cyclic lactams; alkyl aldehydes or alkyl halides and alkylamines which may condense and be reduced to form an alkyl amine bridge; or side chains which may be connected to form an alkyl, alkenyl, alkynyl, ether or thioether bond. Preferred alkyl chains include lower alkyl groups having from about 1 to about 6 carbon atoms.

An additional aspect of the present invention is directed to agonist analogues of SEQ ID NO:2 which are not bridged, and wherein X and Y are independently selected from Ala, Ser, Cys, Val, Leu and Ile or alkyl, aryl, or aralkyl esters and ethers of Ser or Cys.

Biologically active derivatives of the above agonist analogues are also included within the scope of this invention in which the stereochemistry of individual amino acids may be inverted from (L)/S to (D)/R at one or more specific sites.

Also included within the scope of this invention are the agonist analogs modified by glycosylation of Asn, Ser and/or Thr residues.

Biologically active agonist analogs of amylin are included within the scope of this invention which contain less peptide character. Such peptide mimetics may include, for example, one or more of the following substitutions for —CO—NH— amide bonds: depsipeptides (—CO—O—), iminomethylenes (—CH₂—NH—), trans-alkenes (—CH═CH—), beta-enaminonitriles (—C(═CH—CN)—NH—), thioamides (—CS—NH—), thiomethylenes (—S—CH₂— or —CH₂—S—), methylenes (—CH₂—C₂—) and retro-amides (—NH—CO—).

Compounds of this invention form salts with various inorganic and organic acids and bases. Such salts include salts prepared with organic and inorganic acids, for example, HCl, HBr, H₂SO₄, H₃PO₄, trifluoroacetic acid, acetic acid, formic acid, methanesulfonic acid, toluenesulfonic acid, maleic acid, fumaric acid and camphorsulfonic acid. Salts prepared with bases include, for example, ammonium salts, alkali metal salts (such as sodium and potassium salts) and alkali earth salts (such as calcium and magnesium salts). Acetate, hydrochloride, and trifluoroacetate salts are preferred.

Exemplary compounds include, but are not limited to des-¹Lys-h-amylin, ²⁸Pro-h-amylin, ^{25,28,29}Pro-h-amylin, ¹⁸Arg^{25,28}Pro-h-amylin, and des-¹Lys¹⁸Arg^{25,28}Pro-h-amylin, all show amylin activity in vivo in treated test animals, (e.g., provoking marked hyperlactemia followed by hyperglycemia). In addition to having activities characteristic of amylin, certain of the preferred compounds of the invention have also been found to possess more desirable solubility and stability characteristics when compared to human amylin. Examples of these compounds include ²⁵Pro²⁶Val^{28,29} Pro-h-amylin, ^{25,28,29}Pro-h-amylin, and ¹⁸Arg^{25,28}Pro-h-amylin.

Other compounds include ¹⁸Arg^{25,28}Pro-h-amylin, des-¹Lys¹⁸Arg^{25,28}Pro-h-amylin, ¹⁸Arg^{25,28,29}Pro-h-amylin, des-¹Lys¹⁸Arg^{25,28,29}Pro-h-amylin, ^{25,28,29}Pro-h-amylin, des-¹Lys^{25,28,29}Pro-h-amylin, ²⁵Pro²⁶Val^{28,29}Pro-h-amylin, ²³Leu²⁵Pro²⁶Val^{28,29}Pro-h-amylin, ²³Leu²⁵Pro²⁶Val²⁸Pro-h-amylin, des-¹Lys²³Leu²⁵Pro²⁶Val²⁸Pro-h-amylin, ¹⁸Arg²³Leu²⁵Pro²⁶Val.²⁸Pro-h-amylin, ¹⁸Arg²³Leu^{25,28,29}Pro-h-amylin, ¹⁸Arg²³Leu^{25,28}Pro-h-amylin, ¹⁷Ile²³Leu^{25,28,29}Pro-h-amylin, ¹⁷Ile^{25,28,29}Pro-h-amylin, des-¹Lys¹⁷Ile²³Leu^{25,28,29}Pro-h-amylin, ¹⁷Ile¹⁸Arg²³Leu-h-amylin, ¹⁷Ile¹⁸Arg²³Leu²⁶Val²⁹Pro-h-amylin, ¹⁷Ile¹⁸Arg²³Leu²⁵Pro²⁶Val^{28,29}Pro-h-amylin, ¹³Thr²¹His²³Leu²⁶Ala²⁸Leu²⁹Pro³¹Asp-h-amylin, ¹³Thr²¹His²³Leu²⁶Ala²⁹Pro³¹Asp-h-amylin, des-¹Lys¹³Thr²¹His²³Leu²⁶Ala²⁸Pro³¹Asp-h-amylin, ¹³Thr¹⁸Arg²¹His²³Leu²⁶Ala²⁹Pro³¹Asp-h-amylin, ¹³Thr¹⁸Arg²¹His²³Leu^{28,29}Pro³¹Asp-h-amylin, and ¹³Thr¹⁸Arg²¹His²³Leu²⁵Pro²⁶Ala^{28,29}Pro³¹Asp-h-amylin.

Useful amylin agonist analogs include those identified in an International Application, WPI Acc. No. 93-182488/22, entitled “New Amylin Agonist Peptides Used for Treatment and Prevention of Hypoglycemia and Diabetes Mellitus,” the contents of which is also hereby incorporated by reference.

Amylin agonists useful in the invention may also include fragments of amylin and its analogs as described above as well as those described in EP 289287, the contents of which are herein incorporated by reference. Amylin agonists may also be compounds having at least 60, 65, 70, 75, 80, 85, 90, 95, or 99% amino acid sequence identity to SEQ ID NO:1 having amylin activity. Amylin agonists also include small molecules, non-peptide molecules, for example those based on small molecule chemistry. “Amylin activity” as used herein includes the ability of amylin to affect ghrelin levels in a body. Amylin agonists also include analogs of amylin having insertions, deletions, extensions and/or substitutions in at least one or more amino acid positions of SEQ ID NO:1. The number of amino acid insertions, deletions, or substitutions may be at least 5, 10, 15, 20, 25, or 30. Insertions, extensions,or substitutions may be with other natural amino acids, synthetic amino acids, peptidomimetics, or other chemical compounds. Amylin agonists, as contemplated in the invention may also be calcitonins, such as teleost calcitonins, and their analogs, as well as calcitonin-gene-related peptides (CGRP) and their analogs.

In general, amylin agonists or amylin agonist analogs are recognized as referring to compounds which, by directly or indirectly interacting or binding with one or more receptors, mimics an action of amylin. Conversely, amylin antagonists by directly or indirectly interacting or binding with one or more receptors, suppresses an action of amylin. Such interactions or binding events include those that affect ghrelin levels.

Amylin antagonists contemplated in the use of the invention include AC66 (sCT[8-32]) and derivatives such as AC187 (Ac ³⁰Asn, ³²Tyr-sCT[8-32]) a 25 amino acid peptide fragment of salmon calcitonin, developed as a selective amylin receptor antagonist over CGRP receptors. Other useful antagonists include antagonists described in U.S. Pat. Nos. 5,625,032 and 5,580,953, which are incorporated herein by reference. Such compounds include:

X—R₁-Thr-Gln-R₂-Leu-Ala-Asn-R₃-Leu-Val-Arg-Leu-Gln-Thr-Tyr-Pro-Arg-Thr-Asn-Val-Gly-R₄-Asn-Thr-Tyr-NH₂ (SEQ ID NO:3)
R₁ is Ala or a bond;
R₂ is Arg, Gln, Lys, Asn or Leu;
R₃ is Gln, Glu, Asn, Asp or Phe;
R₄ is Ala or Ser; and
X is hydrogen or an acetyl group.

Methods of testing compounds for amylin activity are known in the art. Exemplary screening methods and assays for testing amylin agonists or antagonists are described in the Examples, particularly Example 4 herein, and in U.S. Pat. Nos. 5,264,372 and 5,686,411, which are incorporated herein by reference.

Activity as amylin agonists and/or analogs can be confirmed and quantified by performing various screening assays, including the nucleus accumbens receptor binding assay, followed by the soleus muscle assay, a gastric emptying assay, or by the ability to induce hypocalcemia or reduce postprandial hyperglycemia in mammals.

The receptor binding assay, a competition assay that measures the ability of compounds to bind specifically to membrane-bound amylin receptors, is described in U.S. Pat. Nos. 5,264,372 and 5,686,411, the disclosures of which are incorporated herein by reference. A preferred source of the membrane preparations used in the assay is the basal forebrain which comprises membranes from the nucleus accumbens and surrounding regions. Compounds being assayed compete for binding to these receptor preparations with ¹²⁵I Bolton Hunter rat amylin. Competition curves, wherein the amount bound (B) is plotted as a function of the log of the concentration of ligand, are analyzed by computer using analyses by nonlinear regression to a 4-parameter logistic equation (Inplot program; GraphPAD Software, San Diego, Calif.) or the ALLFIT program of DeLean et al. (ALLFIT, Version 2.7 (NIH, Bethesda, Md. 20892)). Munson and Rodbard, Anal. Biochem. 107:220-239 (1980).

Assays of biological activity of amylin agonists/analogs in the soleus muscle may be performed using previously described methods (Leighton, B. and Cooper, Nature, 335:632-635 (1988); Cooper, et al., Proc. Natl. Acad. Sci. USA 85:7763-7766 (1988)), in which amylin agonist activity may be assessed by measuring the inhibition of insulin-stimulated glycogen synthesis. In brief, an exemplary method includes soleus muscle strips prepared from 12-h fasted male Wistar rats. The tendons of the muscles are ligated before attachment to stainless steel clips. Muscle strips are pre-incubated in Erlenmeyer flasks containing 3.5 ml Krebs-Ringer bicarbonate buffer, 7 mM N-2-hydroxyethyl-peperazine-N′-2-ethane-sulphonic acid, pH 7.4, and 5.5 mM pyruvate. Flasks are sealed and gassed continuously with O₂ and CO₂ in the ratio 19:1 (v/v). After pre-incubation of muscles in this medium for 30 min at 37° C. in an oscillating water bath, the muscles strips are transferred to similar vials containing identical medium (except pyruvate) with added [U—¹⁴C] glucose (0.5 μCi/ml) and insulin (100 μU/ml). The flasks are sealed and re-gassed for an initial 15 min in a 1-h incubation. At the end of the incubation period, muscles are blotted and rapidly frozen in liquid N₂. The concentration of lactate in the incubation medium can be determined spectrophotometrically and [U—¹⁴C]glucose incorporation in glycogen measured. Amylin antagonist activity is assessed by measuring the resumption of insulin-stimulated glycogen synthesis in the presence of 100 nM rat amylin and an amylin antagonist.

Methods of measuring the rate of gastric emptying are disclosed in, for example, Young et al. In a phenol red method, conscious rats receive by gavage an acoloric gel containing methyl cellulose and a phenol red indicator. Twenty minutes after gavage, animals are anesthetized using halothane, the stomach exposed and clamped at the pyloric and lower esophageal sphincters, removed and opened into an alkaline solution. Stomach content may be derived from the intensity of the phenol red in the alkaline solution, measured by absorbance at a wavelength of 560 nm. In a tritiated glucose method, conscious rats are gavaged with tritiated glucose in water. The rats are gently restrained by the tail, the tip of which is anesthetized using lidocaine. Tritium in the plasma separated from tail blood is collected at various timepoints and detected in a beta counter. Test compounds are normally administered about one minute before gavage.

Amylin agonist and antagonist compounds may exhibit activity in the receptor binding assay on the order of less than about 1 to 5 nM, preferably less than about 1 nM and more preferably less than about 50 pM. In the soleus muscle assay, amylin agonist compounds may show EC₅₀ values on the order of less than about 1 to 10 micromolar. In the soleus muscle assay, amylin antagonists may show IC₅₀ values on the order of less than about 1 to 2 micro molar. In the gastric emptying assays, preferred agonist compounds show ED₅₀ values on the order of less than 100 μg/rat. Antagonist compounds would show no effect or the opposite effect in the gastric emptying assay.

In one exemplary method of making the compounds, amylin, amylin agonists and analogs, and amylin antagonists may be prepared using standard solid-phase peptide synthesis techniques and preferably an automated or semiautomated peptide synthesizer. Typically, using such techniques, an α-N-carbamoyl protected amino acid and an amino acid attached to the growing peptide chain on a resin are coupled at room temperature in an inert solvent such as dimethylformamide, N-methylpyrrolidinone or methylene chloride in the presence of coupling agents such as dicyclohexylcarbodiimide and 1-hydroxybenzotriazole in the presence of a base such as diisopropylethylamine. The α-N-carbamoyl protecting group is removed from the resulting peptide-resin using a reagent such as trifluoroacetic acid or piperidine, and the coupling reaction repeated with the next desired N-protected amino acid to be added to the peptide chain. Suitable N-protecting groups are well known in the art, with t-butyloxycarbonyl (tBoc) and fluorenylmethoxycarbonyl (Fmoc) being preferred herein. Other methods of synthesizing or expressing amylin and amylin agonists and purifying them are known to the skilled artisan.

Dosage/Formulation

Amylin, amylin agonist, or amylin antagonists (herein referred to as the “compounds”) may be administered alone or in combination with pharmaceutically acceptable carriers or excipients, in either single or multiple doses. These pharmaceutical compounds may be formulated with pharmaceutically acceptable carriers or diluents as well as any other known adjuvants and excipients in accordance with conventional techniques such as those disclosed in Remington's Pharmaceutical Sciences by E. W. Martin. See also Wang, Y. J. and Hanson, M. A. “Parenteral Formulations of Proteins and Peptides: Stability and Stabilizers,” Journal of Parenteral Science and Technology, Technical Report No. 10, Supp. 42:2S (1988).

In some cases where ghrelin suppression is desired, it may be convenient to provide the compound with insulin or glucose (or a source of glucose), as these compounds may have some ghrelin suppression activity, in a single composition or solution for administration together or separately (in time and/or site of administration). An example is those patients requiring insulin therapy in whom it has been shown that combined insulin plus amylin replacement therapy (for example, with the amylin agonist pramlintide) can have metabolic benefits over those obtained from therapy with insulin alone. There are, however, contraindications to the use of insulin and glucose with certain patients and disease states. A suitable administration format may best be determined by a medical practitioner for each patient individually.

Exemplary formulations for an amylin, amylin agonist, or amylin antagonist can be found in U.S. Pat. No. 6,410,511 and U.S. patent application Ser. No. 10/159,779, filed May 31, 2002, which are incorporated herein by reference.

In general, amylin, amylin agonists, or amylin antagonists may be formulated into a stable, safe pharmaceutical composition for administration to a patient. Pharmaceutical formulations contemplated for use in the methods of the invention may comprise approximately 0.01 to 1.0% (w/v), preferably 0.05 to 1.0%, of an amylin, amylin agonist, or amylin antagonist, approximately 0.02 to 0.5% (w/v) of an acetate, phosphate, citrate or glutamate buffer allowing a pH of the final composition of from about 3.0 to about 7.0; approximately 1.0 to 10% (w/v) of a carbohydrate or polyhydric alcohol tonicifier and, optionally, approximately 0.005 to 1.0% (w/v) of a preservative selected from the group consisting of m-cresol, benzyl alcohol, methyl, ethyl, propyl and butyl parabens and phenol. Such a preservative is generally included if the formulated peptide is to be included in a multiple use product.

In a particular embodiment of the present invention, a pharmaceutical formulation of the present invention may contain a range of concentrations of amylin, amylin agonist, or amylin antagonist, e.g., between about 0.01% to about 98% w/w, or between about 1 to about 98% w/w, or preferably between 80% and 90% w/w, or preferably between about 0.01% to about 50% w/w, or more preferably between about 10% to about 25% w/w in this embodiment. A sufficient amount of water for injection may be used to obtain the desired concentration of solution.

Additional tonicifying agents such as sodium chloride, as well as other known excipients, may also be present, if desired. It is preferred, however, if such excipients maintain the overall tonicity of the amylin, amylin agonist, or amylin antagonist. An excipient may be included in the presently described formulations at various concentrations. For example, an excipient may be included in the concentration range from about 0.02% to about 20% w/w, preferably between about 0.02% and 0.5% w/w, about 0.02% to about 10% w/w, or about 1% to about 20% w/w. In addition, similar to the present formulations themselves, an excipient may be included in solid (including powdered), liquid, semi-solid or gel form.

The pharmaceutical formulations may be composed in various forms, e.g., solid, liquid, semisolid or liquid. The term “solid”, as used herein, is meant to encompass all normal uses of this term including, for example, powders and lyophilized formulations. The presently described formulations may be lyophilized.

The terms buffer, buffer solution and buffered solution, when used with reference to hydrogen-ion concentration or pH, refer to the ability of a system, particularly an aqueous solution, to resist a change of pH on adding acid or alkali, or on dilution with a solvent. Characteristic of buffered solutions, which undergo small changes of pH on addition of acid or base, is the presence either of a weak acid and a salt of the weak acid, or a weak base and a salt of the weak base. An example of the former system is acetic acid and sodium acetate. The change of pH is slight as long as the amount of hydronium or hydroxyl ion added does not exceed the capacity of the buffer system to neutralize it.

As described herein, a variety of liquid vehicles are suitable for use in the present peptide formulations, for example, water or an aqueous/organic solvent mixture or suspension.

The stability of a peptide formulation of the present invention is enhanced by maintaining the pH of the formulation in the range of about 3.0 to about 7.0 when in liquid form. Preferably, the pH of the formulation is maintained in the range of about 3.5 to 5.0, or about 3.5 to 6.5, most preferably from about 3.7 to 4.3, or about 3.8 to 4.2. A frequently preferred pH may be about 4.0. While not seeking to be bound by this theory, it is presently understood that where the pH of the pharmaceutical formulation exceeds 5.5, chemical degradation of the peptide may be accelerated such that the shelf life is less than about two years.

The buffer used in the practice of the present invention is an acetate buffer (preferably at a final formulation concentration of from about 1-5 to about 60 mM), phosphate buffer (preferably at a final formulation concentration of from about 1-5 to about 30 mM) or glutamate buffer (preferably at a final formulation concentration of from about 1-5 to about 60 mM). The most preferred buffer is acetate (preferably at a final formulation concentration of from about 5 to about 30 mM).

A stabilizer may be included in the present formulation but, and importantly, is not necessarily needed. If included, however, a stabilizer useful in the practice of the present invention is a carbohydrate or a polyhydric alcohol. A suitable stabilizer useful in the practice of the present invention is approximately 1.0 to 10% (w/v) of a carbohydrate or polyhydric alcohol. The polyhydric alcohols and carbohydrates share the same feature in their backbones, i.e., —CHOH—CHOH—, which is responsible for stabilizing the proteins. The polyhydric alcohols include such compounds as sorbitol, mannitol, glycerol, and polyethylene glycols (PEGs). These compounds are straight-chain molecules. The carbohydrates, such as mannose, ribose, sucrose, fructose, trehalose, maltose, inositol, and lactose, on the other hand, are cyclic molecules that may contain a keto or aldehyde group. These two classes of compounds have been demonstrated to be effective in stabilizing protein against denaturation caused by elevated temperature and by freeze-thaw or freeze-drying processes. Suitable carbohydrates include: galactose, arabinose, lactose or any other carbohydrate which does not have an adverse affect on a diabetic patient, i.e., the carbohydrate is not metabolized to form unacceptably large concentrations of glucose in the blood. Such carbohydrates are well known in the art as suitable for diabetics. Sucrose and fructose are suitable for use with amylin, amylin agonist, or amylin antagonist in non-diabetic applications (e.g. treating obesity).

Preferably, if a stabilizer is included, the amylin, amylin agonist, or amylin antagonist is stabilized with a polyhydric alcohol such as sorbitol, mannitol, inositol, glycerol, xylitol, and polypropylene/ethylene glycol copolymer, as well as various polyethylene glycols (PEG) of molecular weight 200, 400, 1450, 3350, 4000, 6000, and 8000). Mannitol is the preferred polyhydric alcohol. Another useful feature of the lyophilized formulations of the present invention is the maintenance of the tonicity of the lyophilized formulations described herein with the same formulation component that serves to maintain their stability. Mannitol is the preferred polyhydric alcohol used for this purpose.

The United States Pharmacopeia (USP) states that anti-microbial agents in bacteriostatic or fungistatic concentrations must be added to preparations contained in multiple dose containers. They must be present in adequate concentration at the time of use to prevent the multiplication of microorganisms inadvertently introduced into the preparation while withdrawing a portion of the contents with a hypodermic needle and syringe, or using other invasive means for delivery, such as pen injectors. Antimicrobial agents should be evaluated to ensure compatibility with all other components of the formula, and their activity should be evaluated in the total formula to ensure that a particular agent that is effective in one formulation is not ineffective in another. It is not uncommon to find that a particular antimicrobial agent will be effective in one formulation but not effective in another formulation.

A preservative is, in the common pharmaceutical sense, a substance that prevents or inhibits microbial growth and may be added to pharmaceutical formulations for this purpose to avoid consequent spoilage of the formulation by microorganisms. While the amount of the preservative is not great, it may nevertheless affect the overall stability of the peptide.

While the preservative for use in the pharmaceutical compositions can range from 0.005 to 1.0% (w/v), the preferred range for each preservative, alone or in combination with others, is: benzyl alcohol (0.1-1.0%), or m-cresol (0.1-0.6%), or phenol (0.1-0.8%) or combination of methyl (0.05-0.25%) and ethyl or propyl or butyl (0.005%-0.03%) parabens. The parabens are lower alkyl esters of para-hydroxybenzoic acid.

A detailed description of each preservative is set forth in “Remington's Pharmaceutical Sciences” as well as Pharmaceutical Dosage Forms: Parenteral Medications, Vol. 1, 1992, Avis et al.

Pramlintide, human 25, 28, 29 Pro-amylin does not have a tendency to adsorb onto the glass in a glass container when in a liquid form, therefore, a surfactant is not required to further stabilize the pharmaceutical formulation. However, with regard to amylin, amylin agonist, or amylin antagonist which do have such a tendency when in liquid form, a surfactant should be used in their formulation. These formulations may then be lyophilized. Surfactants frequently cause denaturation of protein, both of hydrophobic disruption and by salt bridge separation. Relatively low concentrations of surfactant may exert a potent denaturing activity, because of the strong interactions between surfactant moieties and the reactive sites on proteins. However, judicious use of this interaction can stabilize proteins against interfacial or surface denaturation. Surfactants which could further stabilize the peptide may optionally be present in the range of about 0.001 to 0.3% (w/v) of the total formulation and include polysorbate 80 (i.e., polyoxyethylene(20) sorbitan monooleate), CHAPS® (i.e., 3-[(3-cholamidopropyl)dimethylammonio] 1-propanesulfonate), Brij® (e.g., Brij 35, which is (polyoxyethylene (23) lauryl ether), poloxamer, or another non-ionic surfactant.

It may also be desirable to add sodium chloride or other salt to adjust the tonicity of the pharmaceutical formulation, depending on the tonicifier selected. However, this is optional and depends on the particular formulation selected. Parenteral formulations preferably may be isotonic or substantially isotonic.

A preferred vehicle for parenteral products is water. Water of suitable quality for parenteral administration can be prepared either by distillation or by reverse osmosis. Water for injection is the preferred aqueous vehicle for use in the pharmaceutical formulations.

It is possible that other ingredients may be present in the pharmaceutical formulations. Such additional ingredients may include, e.g., wetting agents, emulsifiers, oils, antioxidants, bulking agents, tonicity modifiers, chelating agents, metal ions, oleaginous vehicles, proteins (e.g., human serum albumin, gelatin or proteins) and a zwitterion (e.g., an amino acid such as betaine, taurine, arginine, glycine, lysine and histidine). Additionally, polymer solutions, or mixtures with polymers provide the opportunity for controlled release of the peptide. Such additional ingredients, of course, should not adversely affect the overall stability of the pharmaceutical formulation of the present invention.

Containers are also an integral part of the formulation of an injection and may be considered a component, for there is no container that is totally inert, or does not in some way affect the liquid it contains, particularly if the liquid is aqueous. Therefore, the selection of a container for a particular injection must be based on a consideration of the composition of the container, as well as of the solution, and the treatment to which it will be subjected. Adsorption of the peptide to the glass surface of the vial can also be minimized, if necessary, by use of borosilicate glass, for example, Wheaton Type I borosilicate glass #33 (Wheaton Type I-33) or its equivalent (Wheaton Glass Co.). Other vendors of similar borosilicate glass vials and cartridges acceptable for manufacture include Kimbel Glass Co., West Co., Bünder Glas GMBH and Forma Vitrum. The biological and chemical properties of amylin may be stabilized by formulation and lyophilization in a Wheaton Type I-33 borosilicate serum vial to a final concentration of 0.1 mg/ml and 10 mg/ml of amylin in the presence of 5% mannitol, and 0.02% Tween 80.

In order to permit introduction of a needle from a hypodermic syringe into a multiple-dose vial and provide for resealing as soon as the needle is withdrawn, the open end of each vial is preferably sealed with a rubber stopper closure held in place by an aluminum band.

Stoppers for glass vials, such as, West 4416/50, 4416/50 (Teflon faced) and 4406/40, Abbott 5139 or any equivalent stopper can be used as the closure for pharmaceutical for injection. These stoppers are compatible with the peptide as well as the other components of the formulation. The inventors have also discovered that these stoppers pass the stopper integrity test when tested using patient use patterns, e.g., the stopper can withstand at least about 100 injections. Alternatively, the peptide can be lyophilized in to vials, syringes or cartridges for subsequent reconstitution. Liquid formulations of the present invention can be filled into one or two chambered cartridges, or one or two chamber syringes.

Each of the components of the pharmaceutical formulation described above is known in the art and is described in Pharmaceutical Dosage Forms: Parenteral Medications, Vol. 1, 2nd ed., Avis et al. Ed., Mercel Dekker, New York, N.Y. 1992, which is incorporated by reference in its entirety herein.

The manufacturing process for the above liquid formulations generally involves compounding, sterile filtration and filling steps. The compounding procedure involves dissolution of ingredients in a specific order (preservative followed by stabilizer/tonicity agents, buffers and peptide) or dissolving at the same time.

Alternative formulations, e.g., non-parenteral, may not require sterilization. However, if sterilization is desired or necessary, any suitable sterilization process can be used in developing the peptide pharmaceutical formulation of the present invention. Typical sterilization processes include filtration, steam (moist heat), dry heat, gases (e.g., ethylene oxide, formaldehyde, chlorine dioxide, propylene oxide, beta-propiolacctone, ozone, chloropicrin, peracetic acid methyl bromide and the like), exposure to a radiation source, and aseptic handling. Filtration is the preferred method of sterilization for liquid formulations of the present invention. The sterile filtration involves filtration through 0.45 μm and 0.22 μm (1 or 2) which may be connected in series. After filtration, the solution is filled into appropriate vials or containers.

The liquid pharmaceutical formulations of the present invention are intended for parenteral administration. Suitable routes of administration include intramuscular, intravenous, subcutaneous, intradermal, intraarticular, intrathecal and the like. The subcutaneous route of administration is preferred. Mucosal delivery is also preferred. These routes include, but are not limited to, oral, nasal, sublingual, pulmonary and buccal routes which may include administration of the peptide in liquid, semi-solid or solid form. Administration via these routes requires substantially more peptide to obtain the desired biological effects due to decreased bioavailability compared to parenteral delivery. In addition, parenteral controlled release delivery can be achieved by forming polymeric microcapsules, matrices, solutions, implants and devices and administering them parenterally or by surgical means. Examples of controlled release formulations are described in U.S. Pat. Nos. 6,368,630, 6,379,704, and 5,766,627, which are incorporated herein by reference. These dosage forms may have a lower bioavailability due to entrapment of some of the peptide in the polymer matrix or device. See e.g., U.S. Pat. Nos. 6,379,704, 6,379,703, and 6,296,842.

The compounds may be provided in dosage unit form containing an amount of the compound with or without insulin or glucose (or a source of glucose) that will be effective in one or multiple doses to control the effects of ghrelin. Therapeutically effective amounts of the compounds for the treatment of ghrelin-associated diseases or disorders are those sufficient to treat, prevent, or ameliorate the physiological effects of undesirable levels of ghrelin. As will be recognized by those in the field, an effective amount of therapeutic agent will vary with many factors including the age and weight of the patient, the patient's physical condition, the condition to be treated, and other factors.

However, typical doses may contain from a lower limit of about 1 μg, 5 μg, 10 μg, 50 μg to 100 μg to an upper limit of about 100 μg, 500 μg, 1 mg, 5 mg, or 10 mg of the pharmaceutical compound per day for amylin-deficient patients. Lower limits for hyperamylinemic patients may be about 10 μg, 50 μg, 100 μg, 500 μg, or 1 mg and upper limits may be 1 mg, 5 mg, 10 mg, 50 mg or 100 mg of the pharmaceutical compound per day. Also contemplated are other dose ranges such as 0.1 μg to 1 mg of the compound per dose. The doses per day may be delivered in discrete unit doses, provided continuously in a 24 hour period or any portion of that the 24 hours. The number of doses per day may be from 1 to about 4 per day, although it could be more. Continuous delivery can be in the form of continuous infusions. Exemplary doses and infusion rates include from 0.005 nmol/kg to about 20 nmol/kg per discrete dose or from about 0.01/pmol/kg/min to about 10 pmol/kg/min in a continuous infusion. These doses and infusions can be delivered by intravenous administration (i.v.) or subcutaneous administration (s.c.). Exemplary total dose/delivery of the pharmaceutical composition given i.v. may be about 2 μg to about 8 mg per day, whereas total dose/delivery of the pharmaceutical composition given s.c may be about 6 μg to about 6 mg per day.

While the foregoing description discloses the present invention, with examples provided for the purpose of illustration, it will be understood that the practice of the present invention encompasses all of the usual variations, adaptations, or modifications as being within the scope of the claimed invention.

1-3. (canceled)

4. The method according to claim 14 wherein the disease or disorder is Prader-Willi syndrome, diabetes mellitus, complications of diabetes mellitus, obesity, hyperphagia, or hypernutrition.

5-11. (canceled)

12. A method of modulating ghrelin levels in an individual comprising administering a composition comprising an amylin, an amylin agonist, an amylin antagonist, or a compound to induce or inhibit endogenous amylin levels to an individual in need of modulating ghrelin levels in an amount effective to modulate ghrelin levels in the individual.

13. A method of reducing ghrelin levels in an individual comprising administering to an individual in need of reducing ghrelin levels a composition comprising an amylin, an amylin agonist, or an agent that increases endogenous levels of amylin in the individual.

14. A method of ameliorating a disease or disorder that can benefit from a reduction in ghrelin levels comprising administering to an individual having a disease or disorder that can benefit from a reduction in ghrelin levels an effective amount of a composition comprising an amylin, an amylin agonist, or an agent that increases endogenous levels of amylin.

15. The method according to claim 14 wherein the disease or disorder is associated with excessive growth hormone levels.

16. The method according to claim 15 wherein the disease or disorder is acromegaly or retinopathy.

17. The method according to claim 14 further comprising administering at least one of insulin, glucose, or an insulinotropic agent to the individual.

18. The method according to claim 17, wherein the insulintropic agent is at least one of a GLP-1, a GLP-1 analog, a GLP-1 derivative, a GLP-1 agonist, an exendin, an exendin analog, an exendin derivative, an exendin agonist, or a sulfonylurea.

19. A method of increasing ghrelin levels in an individual comprising administering to an individual in need of increased ghrelin levels a composition comprising an amylin antagonist or an agent that decreases endogenous levels of amylin in the individual.

20. A method of ameliorating a disease or disorder that can benefit from an increase in ghrelin levels comprising administering to an individual having a disease or disorder that can benefit from an increase in ghrelin levels an effective amount of a composition comprising an amylin antagonist or an agent that decreases endogenous levels of amylin.

21. The method according to claim 20 wherein the disease or disorder is anorexia nervosa, bulimia, AIDS, wasting, or cachexia.

22. The method according to claim 21 wherein the disease or disorder is cancer cachexia or wasting in the elderly.

23. The method according to claim 20 wherein the disease or disorder is associated with deficient growth hormone levels.
